(19) United States
(12) Patent Application Publication
(10) Pub. No.: US 2014/0142396 A1
(43) Pub. Date: May 22, 2014

(54) Health Measurement Systems

(71) Applicants: Nathan W. Ricks, Lehi, UT (US); Dustin Bybee, Lehi, UT (US); Caleb Manscill, Lehi, UT (US)

(72) Inventors: Nathan W. Ricks, Lehi, UT (US); Dustin Bybee, Lehi, UT (US); Caleb Manscill, Lehi, UT (US)

(21) Appl. No.: 13/871,898

(22) Filed: Apr. 26, 2013

Related U.S. Application Data
(60) Provisional application No. 61/729,078, filed on Nov. 21, 2012.

(51) Int. Cl. A61B 5/0205; G01G 19/50; A61B 5/00; A61B 5/01; A61B 5/145

(52) U.S. Cl. CPC A61B 5/02055; A61B 5/01; A61B 5/14532; A61B 5/14546; A61B 5/14542; A61B 5/0022; A61B 5/4872; A61B 5/681; A61B 5/686; A61B 5/14503; G01G 19/50; A61B 5/021
USPC 600/301

(57) ABSTRACT

A device for monitoring health is disclosed. The device includes a body scale housing, a controller, one or more sensors, and a communications device. The controller receives measurements from the sensors and determined a user's weight and body mass index, and one or more of a user's blood pressure, body temperature, blood glucose levels, C-reactive protein, ketone levels, oxygen saturation, blood cholesterol levels, testosterone levels, and progesterone levels. These measurements are transmitted to an external database over a wireless network.

### CROSS-REFERENCE TO RELATED APPLICATIONS

This application claims benefit to United States Provisional Patent Application Ser. No. 61/368,902 filed Apr. 26, 2013 and also claims benefit to U.S. Provisional Patent Application Ser. No. 61/729,078 filed Nov. 21, 2012.

### BACKGROUND

1. Field of the Invention

The present invention relates generally to the field of health measurement systems and methods. More specifically, the invention relates to a health measurement system, such as a body scale system, for measuring various aspects of an individual's personal health and wellness. Specifically, some implementations of the system measure one or more of an individual's weight, body mass index, body fat percentage, heart rate, heart rate irregularities, and more.

2. Background

The benefits of managing one's weight, eating properly, and maintaining an active lifestyle are well known and researched. These benefits include a low propensity to illness and disease and prolonged life expectancy. Despite these advantages, the general population is experiencing an increase in body weight and a decline in many aspects of its health. One potential cause of this trend is lack of reliable feedback available to specific individuals about various aspects of their health. For instance, while many people understand the need for healthy habits, they often do not recognize the effect of their various daily lifestyle choices and unhealthy habits. Instead, other priorities can divert an individual's focus from health to other cares. As a result, well-intentioned individuals may unintentionally become heavier and less healthy.

To address these challenges, hundreds of fad diets and weight-loss plans promise quick and easy weight loss and good health. These programs try to make weight loss and good health easier, quicker, cheaper, more reliable, and less painful. While these programs can be effective and beneficial, they are often short lived and do not promote long-term health and wellness. These programs can also take an individual's focus off the more fundamental and essential elements of health and wellness, including healthy eating habits, exercise, and physical activity.

Additionally, because many health problems develop over long periods, some people are unaware of the negative effects of their lifestyle choices. For instance, because many individuals feel good or are content with their overall appearance and body weight they fail to recognize or address their unhealthy lifestyle practices. These individuals may later experience serious health problems that may have been avoided if they had understood the effects of their lifestyle choices or if they had been advised of their potential for illness and advised to make preventative changes.

Thus, while current health programs and systems are available, improvements would be desirable.

### SUMMARY

The present invention relates generally to the field of health measurement systems and methods. More specifically, the invention relates to a health measurement system that measures various aspects of an individual's personal health and wellness. The health measurement system can include a body scale that measures a user's body weight and body mass index or body fat percentage. This information can be transmitted across a wireless communications link to a network device that processes the information and provides the user with analytics, advice, encouragement, or other such information via a website or mobile application. The wireless communications link can utilize 3G, GSM, CDMA, or another such wireless communications standard.

Additionally, in some implementations, additional health measurements are acquired by the health measurement system, such as the user's blood pressure, body temperature, glucose levels, C-reactive protein, ketone levels, oxygen saturation levels, cholesterol levels, testosterone levels, progesterone levels, alcohol levels, drug levels, and more. Using this information the health measurement system can recognize health risks and warn and educate the user about potential health risks. For instance, based on the user's measured heart rate, the health measurement system can identify irregularities, such as arrhythmia, bradycardia, or atrial fibrillation. These additional health measurements can be acquired via the body scale or via a separate body attachment.

The separate body attachment device can be worn or carried by the user or attached to a user. The body attachment device can include an anklet, bracelet, armband, clip, finger ring, toe ring, pendant, necklace, or earring or other piercing. Additionally, the separate body attachment device can be implanted in or below the epidermis of the user. The separate body attachment device can include one or more measurement devices for measuring any of the above-mentioned health measurements. For example, the separate body attachment device can measure the user's blood pressure, body temperature, as well as the user's physical activity, movement, and calorie expenditures. These health measurements can be transmitted over a communications link (e.g., a Bluetooth connection) to the body scale, which can process this information and communicate it to the network device.

As mentioned, in some implementations, the user can access these health measurements as well as analytics, graphs, and other information via a website or mobile application. The website and mobile application can access information from the network device and provide this information to the user in an easy-to-understand form. Additionally, the website or mobile application can provide various other forms of health-related information, tools, logs, charts, advice, and warnings. In some instances, the network device can be configured to relay the health measurements to a health professional, health insurance organization, physical trainer, or other third party.

The measurement and presentation of the user's health measurements and information can provide the user with information tools that can assist the user to manage his/her weight and overall health. This system can avoid the downsides of temporary health fads by offering actual, measurable information that can be used to form enduring, effective, and improved lifestyle changes. This information can empower the user to managing his/her weight, eat properly, and maintaining an active lifestyle in order to reduce his/her propensity to illness and disease and prolong his/her life expectancy.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 illustrates a representative health measurement system, according to some embodiments of the present invention.

FIG. 2 illustrates a representative system for implementing embodiments of the invention.

FIG. 3 illustrates a representative body scale, according to some embodiments of the present invention.

FIG. 4 illustrates a representative body attachment, according to some embodiments of the present invention.

FIG. 5 illustrates a representative body scale coupled to a body attachment, according to some embodiments of the present invention.

### DETAILED DESCRIPTION OF THE INVENTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention is grouped into subheadings. The utilization of the subheadings is for convenience of the reader only and is not to be construed as limiting in any sense.

For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).”

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” “in some configurations,” or “in some instances,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

The present invention relates generally to the field of health measurement systems and methods. More specifically, the invention relates to a health measurement system that can measure various aspects of an individual's personal health and wellness.

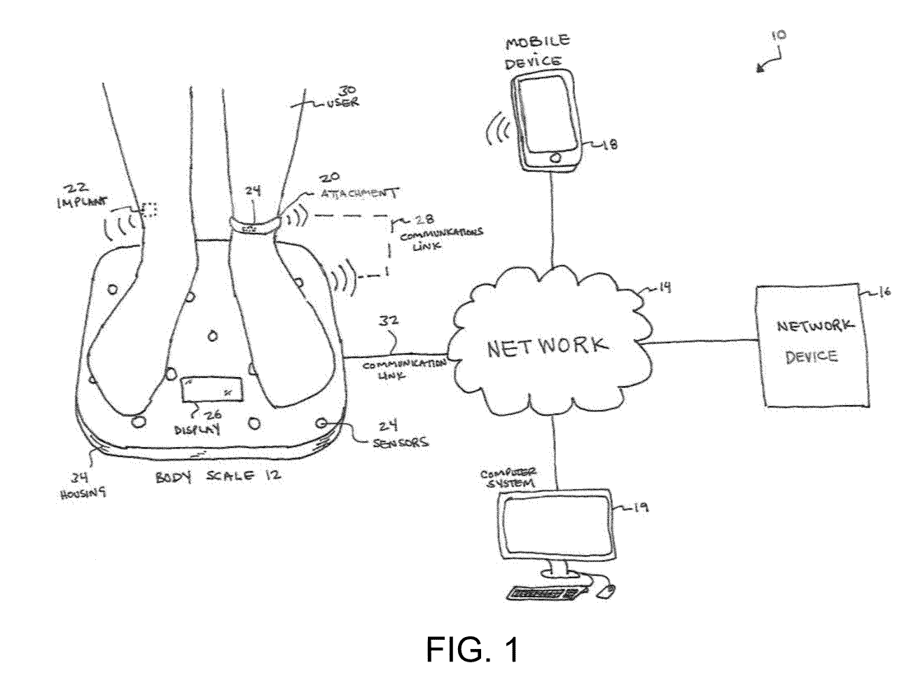

Reference will now be made to FIG. 1, which illustrates a representative health measurement system 100, according to some embodiments. The health measurement system 100 can generally includes a body scale 12, a communications network 14, a network device 16 (e.g., a server, a server system, a computer system, etc.), and optionally a mobile device 18 or computer system 19 and/or a separate body attachment (e.g., the illustrated anklet) 20 or an implant 22. The health measurement system 100 can measure and provide health and wellness measurements and information to a user 30. This information can be provided, at least in part, by a body scale 12. When a user stands on the top surface of the body scale 12, the body scale 12 can measure various aspects of the user's body and health. For example, the body scale 12 can measure body the user's body weight, body mass index or body fat percentage, and/or other health measurements. These health measurements can be made using one or more sensor or measuring devices (herein sensors) disposed on or within a housing 34 of the body scale 34.

Additionally, in some implementations, additional health measurements are acquired by the health measurement system 10, such as the user's blood pressure, body temperature, glucose levels, C-reactive protein, ketone levels, oxygen saturation levels, cholesterol levels, testosterone levels, progesterone levels, alcohol levels, drug levels, and more. Using this information the health measurement system 10 can recognize health risks and warn and educate the user about potential health risks. For instance, based on the user's measured heart rate, the health measurement system 10 can identify irregularities, such as arrhythmia, bradycardia, or atrial fibrillation. These additional health measurements can be acquired via the body scale 12 or via a separate body attachment device (or simply “body attachment”) 20.

The body attachment 20 can be worn or carried by the user. As shown, the body attachment 20 is an anklet. In other embodiments, the body attachment 20 can be a bracelet, clip, finger ring, toe ring, pendant, necklace, earring or other piercing. Additionally, the body attachment 20 can be in the form of an implant 22 that is placed in or below the epidermis of the user 30. The body attachment 20 can include one or more measurement devices for measuring any of the above-mentioned health measurements. For example, in some embodiments, the body attachment 20 can measure the user's blood pressure, body temperature, as well as the user's physical activity, movement, and calorie expenditures. These health measurements can be transmitted over a communications link 28 (e.g., a wired link or a short range wireless link, such as a Bluetooth link) to the body scale 12.

Health measurements can be transmitted by the body scale 12 across a communications link 32, over a communications network 14, to a network device 16. The network device 16 can be a server, a database, and/or other such network devices. The network device 16 can processes and/or analyze the health measurements and provide the user with analytics, advice, encouragement, or other such information via a website or mobile application, which can be accessed via a mobile device 18 (e.g., a mobile phone or tablet computer) or a computer system 19 (e.g., a personal computer, laptop, etc.) For examples, the user's weight, body mass index, blood pressure, body temperature, blood glucose levels, C-reactive protein, ketone levels, oxygen saturation, blood cholesterol levels, testosterone levels, and/or progesterone levels can be accessed through an Internet website and/or a mobile application. The network device 16 can also provide other health related information and services to the user 30. For example, messages, analytics, suggestions, and educational information and services can be provided along with the health measurements. In some embodiments, the health measurement system 10 can compile the user's measurement information and/or additional information and provide the user with an overall health score. In some instance, health-related information and/or health measurements can additionally or alternatively be accessed with the body scale 12.

Each of the respective components of the health measurement system 10 will be described in detail in the sections presented below.

### Body Scale

Specific reference will now be made to the body scale 12 of FIG. 1. As mentioned, the body scale 12 can be configured to take and process measurements, receive measurements from the body attachment 20 or implant 22, and/or transmit information to a network device 16. In some instances, the body scale 12 can functions as both a bathroom scale and serve as a base station that receive and/or transmits health measurements. Generally, the various components of the body scale 12 can be included within a housing 34. The housing 34 can be made of a plastic, metal, glass, composite, or other suitable materials. The housing 34 can contain or support one or more sensors or measuring devices 24, a mass controller, a memory device, a storage device, a display 26, and other such components. In some instances, housing 34 can also contain an internal power source, such as a battery. In other instances, the body scale 12 can acquire power from an external power supply, such as a wall power outlet, to power the body scale 12 or charge the internal power source.

The body scale 12 can acquire health measurements using one or more or other measuring devices 24. Various measurement components can be utilized by the body scale 12, as are discussed in the Health Measurements section below. For example, the body scale 12 can include a weight sensor configured to measure the user's weight when the user 30 stands on the body scale 12.

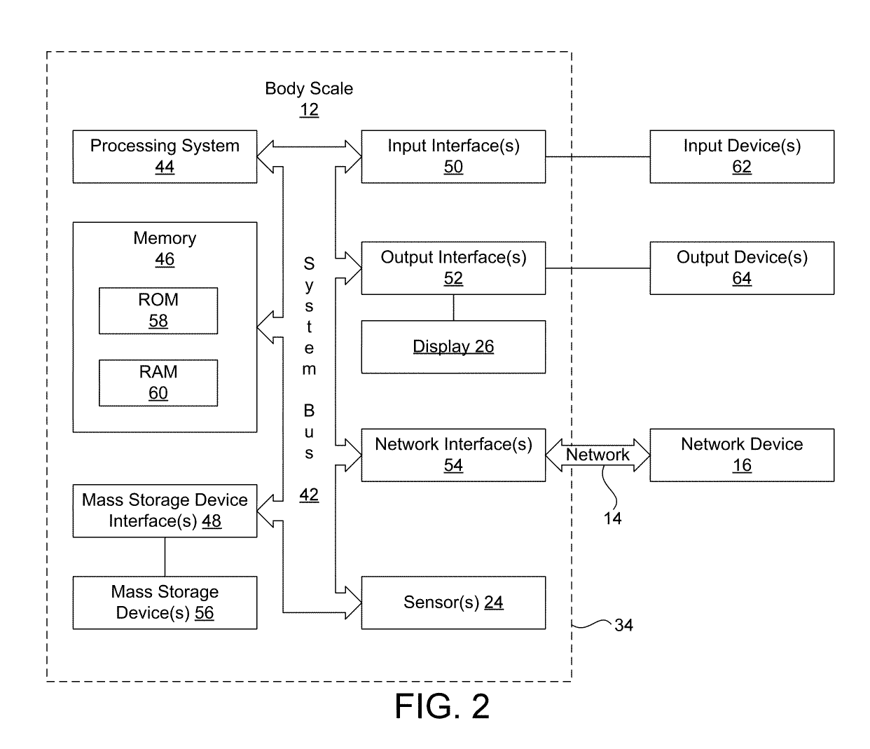

Reference will now be made to FIG. 2, which illustrates a representative operating environment in which embodiments of the body scale 12 may be implemented. Embodiments of the body scale 12 may be practiced by one or more computing devices and in a variety of system configurations. For examples, the body scale 12 can include one or more embedded systems with general purpose processing units, digital/media signal processors (DSP/MSP), application specific integrated circuits (ASIC), stand alone electronic devices, and other such electronic environments.

Embodiments of the body scale 12 can embrace one or more computer-readable media, wherein each medium may be configured to include or includes thereon data or computer executable instructions for calculating body measurement and processing health measurement data. The computer executable instructions can include data structures, objects, programs, routines, or other program modules that may be accessed by a controller 44, for performing body measurement calculations and other identified functions. Computer executable instructions cause the controller 44 to perform a particular function or group of functions and are examples of program code means for implementing steps for methods disclosed herein. Furthermore, a particular sequence of the executable instructions provides an example of corresponding acts that may be used to implement such steps. Examples of computer-readable media include random-access memory (“RAM”) 60, read-only memory (“ROM”) 58, programmable read-only memory (“PROM”), erasable programmable read-only memory (“EPROM”), electrically erasable programmable read-only memory (“EEPROM”), or any other device or component that is capable of providing data or executable instructions that may be accessed by a processing system. While embodiments of the invention embrace the use of all types of computer-readable media, certain embodiments as recited in the claims may be limited to the use of tangible, non-transitory computer-readable media, and the phrases “tangible computer-readable medium” and “non-transitory computer-readable medium” (or plural variations) used herein are intended to exclude transitory propagating signals per se.

As shown in FIG. 2, a representative body scale 12 can includes system bus 42, which may be configured to connect various components thereof and enables data to be exchanged between two or more components. System bus 42 may include one of a variety of bus structures including a memory bus or memory controller, a peripheral bus, or a local bus that uses any of a variety of bus architectures. Typical components connected by system bus 42 include controller 44 and one or more sensors 24. Other components of the body scale 12 can include one or more mass storage device interfaces 48, input interfaces 50, output interfaces 52, and/or network interfaces 54, each of which will be discussed below.

The controller 44 can includes one or more processors, such as a central processor and optionally one or more other processors designed to perform a particular function or task. For example, the controller 44 can be configured to process body measurement data and identify health measurements, potential illnesses, symptoms, or diseases. The controller 44 can also be configured to identify trends in the user's health measurements and present these trends to the user. The controller 44 can also be configured to prepare data measurements for transmission to a network device 16 and process received data from the network device 16. Generally, the controller 44 can execute instructions provided on computer-readable media, such as on memory 46, a magnetic hard disk, a removable magnetic disk, a magnetic cassette, an optical disk, or from a communication connection, which may also be viewed as a computer-readable medium.

The memory 46 includes one or more computer-readable media that may be configured to include or includes thereon data or instructions for manipulating data, and may be accessed by controller 44 through system bus 42. Memory 46 may include, for example, ROM 58, used to permanently store information, and/or RAM 60, used to temporarily store information.

One or more mass storage device interfaces 18 may be used to connect one or more mass storage devices 56 to system bus 42. Various types of data can be stored in the mass storage devices 56, such as a user's name, weight measurement history, other body measurement history, user's height, user's ages, and other demographic and personal information. This information can be used by the controller 44 to identify trends, calculate health measurements (e.g. using body height to calculate body mass index) and to identify potential health risks or symptoms. The mass storage devices 56 may be incorporated into or may be peripheral to the body scale 12 and allow the body scale 12 to retain large amounts of data. Examples of mass storage devices include hard disk drives, solid state storage memory, and flash memory. Mass storage devices 56 and their corresponding computer-readable media provide nonvolatile storage of data and/or executable instructions that may include one or more program modules such as an operating system, one or more application programs, other program modules, or program data. Such executable instructions are examples of program code means for implementing the functions of the body scale 12 disclosed herein.

One or more input interfaces 50 may be employed to enable a user to enter data and/or instructions to the body scale 12 through one or more corresponding input devices 62. Examples of such input devices include a keyboard, touch screen, or buttons. In some embodiments, the input devices 62 are coupled to and/or incorporated into the body scale 12. Similarly, examples of input interfaces 50 that may be used to connect the input devices 62 to the system bus 42 include a serial port, a parallel port, a universal serial bus (“USB”), an integrated circuit, or another interface. For example, in some configurations, the body attachment 20 is electronically coupled to the body scale 12 through an input interfaces 50.

One or more output interfaces 52 may be employed to connect one or more corresponding output devices 64 to system bus 42. Examples of output devices 64 include a monitor or display screen, a speaker, and the like. A particular output device 34 may be integrated with or peripheral to body scale 12. Examples of output interfaces include a video adapter, an audio adapter, a parallel port, and the like.

A display 26 (such as that shown in FIG. 1) can be one representative output device provided by the body scale 12 to present various types of information to the user. For example, the display 26 can display health measurements, such as a body weight, body fat percentage, or other health measurements included in the Health measurements section below. The display 26 can include a touch screen, thus serving the dual function of an input device and an output device.

One or more network interfaces 54 can enable the body scale 12 to exchange information with one or more network devices 16 and/or a body attachment 20. In some embodiments, the body scale 12 includes a first network interface 54 for communicating via a network 14 to one or more network devices 16. The communication link (e.g., link 32 of FIG. 1) that may include hardwired and/or wireless links 32. Examples of network interfaces include a network adapter for connection to a local area network (“LAN”) or a modem, wireless link, or other adapter for connection to a wide area network (“WAN”), such as the Internet. Other examples of a network interface include a network adapter for connection to a cellular telephone network, using a 3G, GSM, CDMA, or another such wireless communications standard. The network interface 54 may be incorporated with or peripheral to body scale 12.

In embodiments utilizing a body attachment 20 (as shown in FIG. 1), the body scale 12 can include a second network interface 54 for communicating with a body attachment 20. Via this communication link (e.g., link 28 of FIG. 1), the body scale 12 can receive health measurements acquired by the body attachment 20. This communication link can be a wireless or direct-wired communication link. For example, in some configurations, this communication link is a short range wireless link (e.g., a Bluetooth link). As such, the body attachment 20 can upload its health measurements to the body scale 12 when the body attachment 20 is in proximity to the body scale 12, such as within 15 feet. Such wireless communication can enable easy and rapid data transmission. In other configurations, this second communication link can be a wired communication link. For example, the body attachment 20 can be connected to the body scale 12 via an electrical connector, such as a USB connector. This wired connection can advantageously charge a battery within the body attachment 20 as data is transmitted between these devices. In other embodiments, body scale 12 and body attachment 20 can be configured to transfer data using Near Field Communication (NFC) standards such as by moving body attachment 20 within the necessary proximity of body scale 12. In such embodiments, the NFC enabled body scale or body attachment can also be configured to communicate with an NFC enabled smart phone or other portable device. Additional information about the body attachment 20 will now be provided.

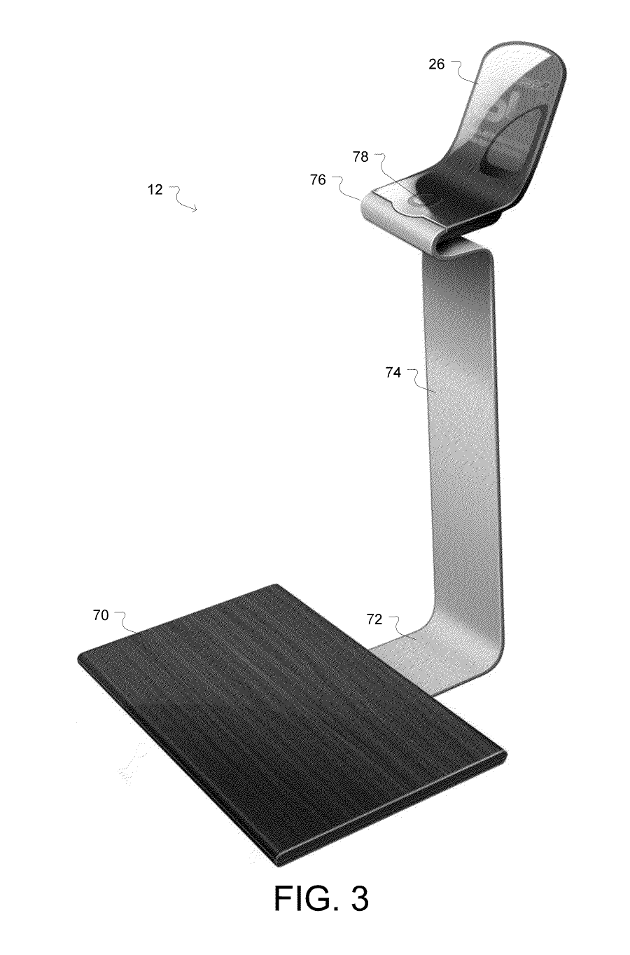

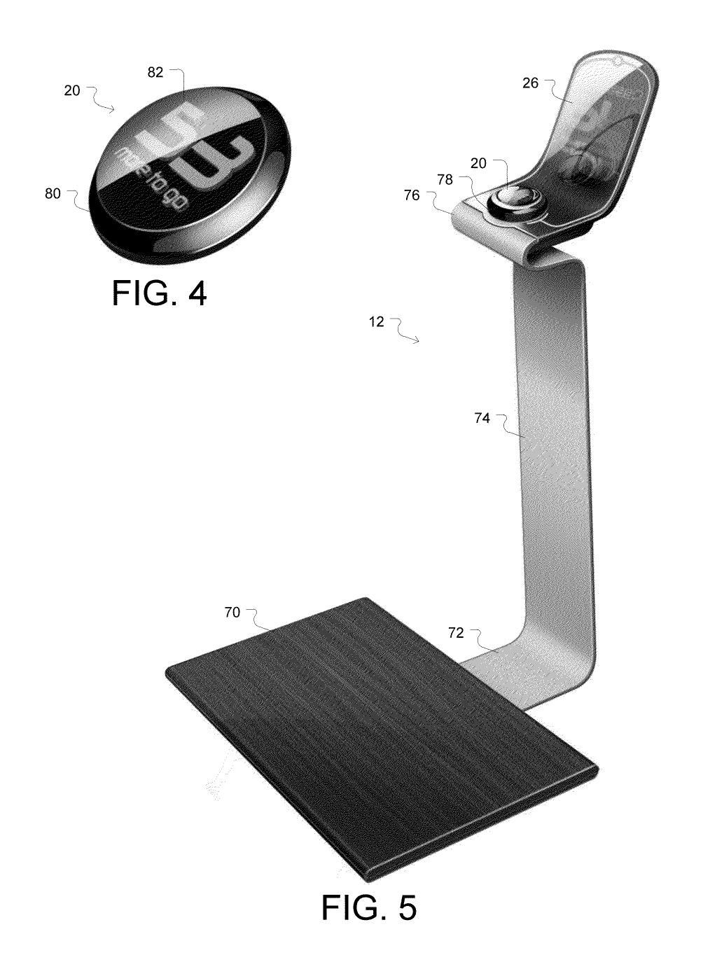

Reference will now be made to FIG. 3, which illustrates alternative embodiments of a body scale 12. As shown, the body scale 12 can include a scale member 70 that can function as a base of the body scale 12. Information measured by the scale member 70 can be displayed on a display 26 that can extend outward and/or upward from the scale member 70. Additionally, the body scale 12 can include a docking station 78 for receiving a body attachment (80, shown in FIG. 4). The scale member 70 can include the one or more sensors (24 shown in FIG. 1) previously described which can be configured to take various measurements from the user when the user is standing on a top surface of the scale member 70. The body scale 12 can be configured to wirelessly transmit and/or receive information to/from a network 14 or network device 16 (as shown in FIG. 1). Accordingly, various body measurements can be displayed to the user via the display 26.

As shown, the display 26 can be elevated above scale member 70 and supported with a riser 74 that physically supports the display 26. The height of the riser 74 can be between approximately 2 feet and approximately 4 feet. The riser 74 can make it easy for a user to look at and interact with the display 26 by placing the display 26 in arms reach of the user when standing on the scale member 70. The riser 74 can extend from the scale member 70 via a horizontal support 72. In some configurations, the horizontal support 72 is capable of telescoping or otherwise adjusting towards and away from the scale member 70 in order to adjust the placement of the display 26 in relation to the user when the user is standing on the scale member 70. The riser 74 can include various bends 76 or other shapes or curves to provide various functions and aesthetic design features to the body scale 12, as shown.

### Body Attachment

Turning again to FIG. 1, specific reference will now be made to the body attachment 20. The body attachment 20 can be an anklet, bracelet, clip, finger ring, toe ring, pendant, necklace, earring or other piercing, implant 22, or other attachable or carry-able device that takes one or more health measurements. These health measurements can include those described in the Health Measurements section below. These health measurements can be taken using one or more sensors 24 and other devices and systems that are also described in the Health Measurements section below. Particularly, the body attachment 24 may be used to acquire health measurements that are more difficult to acquire accurately from the feet. For example, in some embodiments, the body attachment 20 can measure the pulse, body temperature, and/or blood pressure of the user 30.

The body attachment 20 can also be configured to measure the activity level of the user. The user's activity level can provide an indication of health and energy consumption. Activity measurements can be acquired using various sensor and devices, which can be coupled to or incorporated into the body attachment 30. For example, in some embodiments, the body attachment includes one or more of an accelerometer, altimeter, pedometer, tilt sensor, magnetometer, and/or a global positioning system (GPS) device. One or more such device can provide three-dimensional movement and activity measurements to the body attachment 20. The measurements from each of these sensors can be used to recognize changes in a user's location, orientation, altitude, or movement/acceleration. These sensors can also measure the number of steps taken by a user. This information can be used in combined with various algorithms to determine the total amount of energy expended by a user in moving.

In some embodiments, user's activity measurements can be used to measure the number of calories burned by the user 30 in a day or during another period, such as during a workout. In some embodiments, the health measurement system 10 can utilize body temperature measurements, heart rate measurements, and/or motion measurements to calculate or approximate the user's calorie consumption. These calculations can use the total amount of time a user sleeps in a night and differentiate between calorie consumption levels during the daytime and the nighttime.

In some embodiments, user's activity measurements can be used to track a user's exercise activities, including when a user exercises and the duration of the exercise. This information can be used to recognize if the user 30 is adhering to an exercise program. This information can be automatically be logged onto a chart for the user. This information can also be used to transmit reminders, motivational messages, and congratulations to the user 30.

The user's activity measurements can provide information about the user's sleep habits. For example, by identifying when a user stops or slows his/her movements, the health measurement system 10 can identify when the user goes to bed, when the user falls asleep, how often the user wakes up during his/her sleep, and when the user wakes up. By analyzing the information, the health measurement system 10 can identify the duration of the user's sleep and the overall quality of the sleep. By combining such sleep measurements from a single day with measurements over a period, the health measurement system 10 can identify the user's sleep behaviors, patterns, or lack of patterns and provide analysis, information, and sleep suggestions to the user 30.

As mentioned, the body attachment 20 can be electronically coupled to the body scale 12 via a wireless or wired link 28. In some embodiments, the body attachment 20 can include a near field technology chip to allow the body attachment to transfer data to body scale 12 using near field technology standards. In some configurations, the health measuring system includes a dock (not shown) or cradle that can receive the body attachment 20 and electronically couple the body attachment 20 to the body scale 12. The dock can include one or more electrical connections, electrodes, or other suitable connectors for electronically coupling the body attachment 20 to the body scale 12. In some instances, the dock can charge a battery of the body attachment 20 while the body attachment is docked.

Reference will now be made to FIG. 4, but that which illustrates a representative embodiment of the body attachment 20. The body attachment 20 can include housing 80 that poses one or more motion sensors (not shown) and/or a display 82. The display 82 can present various information to a user including the number of steps taken, the number of (estimated or calculated) calories used, the user's body temperature, the user's heart rate, the user's blood pressure, or other body measurements. In some instances, the display 82 can also present the time, the date, the name of the user, or other miscellaneous information. In other instances, the display 82 can also present various media, games, or other entertainment features.

Reference will now be made to FIG. 5, which illustrates the body attachment 20 docked in a docking station 78 of the body scale 12 of FIG. 3. As mentioned, when docked, the body attachment 20 can download or upload information to and from the body scale 12. Additionally, when docked, the body attachment 20 can synchronize user health measurements and data with the body scale 12. For example, when docked, the body attachment 20 can download user activity measurements to the body scale 12. This information can be processed by the body scale 12 or transmitted to a network device (16, shown in FIG. 1) where it is processed. Processed information can be accessed either via the body scale 12, via a mobile device (18, shown in FIG. 1), or via an Internet website. Additionally, this information can be uploaded or synchronized from the body scale 12 to the body attachment 20. Accordingly, the body attachment 20 can both measure body activity and display body measurements to the user.

### Health Measurements

Reference will now be made to the various types of health measurements that can be taken by either the body scale or the body attachment. Reference will also be made to representative sensors or other measuring devices that can be used to take these measurements. It will be understood the body measuring system take health measurements using various sensors or devices, including those not described herein.

In some embodiments, the body measuring system can include one or more sensors used to measure various aspects of a user's body and health. For examples, the body scale can include one or more strain gauge sensors. Strain gauge sensors can employ length sensitive electrical resistance technology or another suitable technology. In some instances, two or more strain gauge sensors can be disposed at supportive locations on the body scale to measure a user's weight accurately despite the user's weight distribution on the body scale.

Once body weight is measured, the body measuring system can calculate the user's body mass index (BMI). In some embodiments, this calculation is performed by the controller of the body scale. BMI is based on the user's weight and height, and provides the user with an indication of whether the user is underweight, normal weight, overweight, or obese. BMI is calculated by dividing the user's weight by the square of his/her height. In some configurations, as previously mentioned, the body scale can receive the user's height from an input device. This information can be stored in a memory or a mass storage device. The body measuring system can then calculate the user's BMI each time the user steps of the scale. This information can be provided to the user automatically or upon request.

Because BMI calculations do not differentiate between fat and not-fat body mass, the body scale can be configured to alternatively or additionally provide a body fat percentage (BF %) measurement, body lean percentage/weight, and/or body water percentage/weight measurements. For example, in some instances, the body scale or body attachment includes two or more electrode pads configured to measure body fat percentage, body lean percentage/weight, and/or body water percentage/weight measurements. With body scale embodiments, when a user stands barefoot on the two or more electrode pads, a small electric current is sent through the user's body. The electric current is sent from a first electrode, up one leg, across the abdomen and down the other leg to the second electrode. The resistance between the electrodes can be used to determine the measure of body fat, body lean mass, and body water, since the resistance can vary between different tissues as fluids in the body. The body measuring system can employ bioelectrical impedance analysis to determine the user's body fat percentage, which is the total weight of the person's body fat divided by the person's weight.

The body measuring system can also use electrode pads or a heart rate monitor to determine a user's heart rate. In some configurations, the heart rate monitor includes an electrocardiogram (ECG or EKG). Heart rate is the number of heartbeats per unit of time (e.g. a minute). Heart rate measurements can assist to determine the level of health of the user and to diagnose and track medical conditions. For instance, healthy or active individuals may have a lower resting heart rate than less active or less healthy individuals. In some configurations, the body measuring system can request that the user be at rest prior to measuring the user's heart rate, to provide a resting heart rate measurement.

In addition to measuring the user's heart rate, the body measuring system can identify any present heart rate abnormalities. For example, the controller of the body scale or the network device can identify arrhythmia, bradycardia, or atrial fibrillation by monitoring the heart rate and regularity. In making these identifications, if the body measuring system identifies that a user's heart rate is to fast the body measuring system can identify or flag a potential arrhythmia condition. If the body measuring system identifies the user's resting heart rate as too slow, such as below beats per minute, the body measuring system can identify or flag a potential bradycardia condition. Additionally, the body measuring system can be configured to identify atrial fibrillation symptoms, in which the user's heart rate is irregular, and which can indicate an increase risk of strokes or other health challenges. When these heart rate irregularities are identified, the body measuring system can provide this information to the user.

In some embodiments, the body measuring system includes a temperature sensor for measuring the user's body temperatures. When disposed on the body scale, the body measuring system can be configured to adjust the measured temperature based on known relationships between feet temperature and average body temperatures because the temperature of the feet can be different than the average body temperature.

In some embodiments, the body measuring system can further includes one or more devices for measuring the user's blood pressure. Blood pressure one of the principle vital signs and can provide useful health information to the user and health professionals. Blood pressure is the pressure that blood exerts upon blood vessel walls within the body. During a heartbeat blood pressure rises from a diastolic pressure to a systolic pressure, each of which can be measured and tracked by the health measurement system. Healthy users may have lower diastolic and systolic blood pressure values. By providing blood pressure measurements to a user, the user can gauge his/her stress levels and progress in maintaining healthy blood pressure levels. In some embodiments, the body measuring system can measure the user's blood pressure using a pulse oximeter or other suitable device. The pulse oximeter can include one or more light sources (e.g. light-emitting diodes) that transmit light into the skin of a patient. The pulse oximeter can also include one or more detectors, such as a photodetector that detects light reflected from an underlying artery. By processing the character of the reflected and the transmitted lights, the pulse oximeter can identify and measure various features of the user's blood, including the user's blood pressure, oxygen saturation, and more.

Various embodiments of the body measuring system can include devices for measuring the user's blood glucose levels. Blood glucose levels, which indicate the amount of glucose in the blood can be important information for users with diabetes. For instance, in some configurations, the body measuring system can incorporate an external glucometer that can interface and communicate with the body measuring system. For example, an external glucometer can communicate with one or more components of the body measuring system via a short range wireless link (e.g., a Bluetooth link). Blood glucose levels can be received, stored, and/or transmitted by the body scale when the external glucometer is located within a minimum range in which the wireless link can be established.

Various embodiments of the body measuring system can include one or more internal or external sensors or other such devices for measuring the user's C-reactive protein levels. C-reactive protein is found the blood and can indicate a rise in inflammation and infection. High levels of C-reactive protein can be problematic to users with heart related diseases. Accordingly, it can be advantageous to monitor the levels of this protein in the blood. In some instances, an external C-reactive protein sensor can communicate with the body measuring system via a short range wireless link. This information can be received, stored, and/or transmitted by the body scale when the external sensor is located within a minimum range in which the wireless link can be established.

In some embodiments, the health measurement system incorporates one or more additional internal or external sensors that can be used to measure ketone levels within the blood. Ketone bodies include water-soluble compounds that are produced in the liver and kidneys. Ketosis occurs when the blood accumulates an excessive level of ketone bodies. This can be cause by eating disorders and low carbohydrate intake. The measurement of ketone levels can assist the user to recognize if his/her ketone levels are in a healthy range or if ketosis is taking place.

In some embodiments, the health measurement system incorporate one or more additional internal or external sensors that can be used to measure blood cholesterol levels, testosterone levels within the blood, progesterone levels within the blood, and/or drug levels within the blood. These measurements can be useful in providing health and fitness related information to the user and for determining the overall health of the user.

It will be understood that other such health measurements known in the art but not described herein can also be taken by the health measurement system using one or more of the body scale and/or the body attachment.

### Network Services

Referring still to FIG. 1, after the body measuring system 10 has acquired health measurements, these measurements can be transmitted from the body scale 12 to a network device 16, over one or more communication links 32 and a communications network 14, such as the Internet. The wireless communications link includes a cellular communications network utilizing, for example, 3G, GSM, CDMA, or another such wireless communications standard. In other instances, the communications link is a wired communications link utilizing, for example, internet protocol (IP) general packet radio service (GPRS) protocol, or other known communications protocols.

In some embodiments, the network device 16 can provide access the health measurements and related information through a website or mobile applications. The website and mobile application can access information from the network device 16 using a mobile device 18 or computer system 19. These interfaces may display health measurements and provide related information in an easy-to-understand form. The website or mobile application can be secured and require user authentication, such as via a user login prior to providing access to user health measurements and information. The website or mobile application can provide a means whereby the user can input information, such as the user's age, height, health history, and health goals. In some configurations, the website or mobile application can provide an interface for the use to submit the type and amount of food that the user consumed in a day, in order to calculate calorie intake.

Additionally, the website or mobile application can provide various other forms of health related information, tools, logs, charts, advice, and warnings. For example, the website or mobile application can chart the user's weight, body fat percentage, or blood pressure over a period of time, such as a month or year. In another example, the website or mobile application can chart the user's heart rate, body temperature, and/or calorie consumption during a period of exercise. In yet another example, the website or mobile application can chart the user's sleep measurements during a time period while providing analysis and suggestions for improved sleep practices. In still another example, the website or mobile application can provide an overview of the user's health, including warning, areas of cautions, and general tips. The website or mobile application can provide one or more interfaces for recording a user's goals, charting the user's progress, and providing goal related messages, reminders, and encouragement.

In some instances, the network device can be configured to relay the health measurements to a health professional, health insurance organization, physical trainer, or other third party. These individuals may be granted access to the user's health measurements and related information by the user or another party. Using this information, these third parties can track a user's general and specific health and provide feedback or analysis of this information. For instance, a doctor can receive periodic updates of the user's cholesterol levels or blood pressure to track the effects of a prescribed treatment plan. In another instance, a physical trainer can receive periodic updates of the user's weight or body fat percentage and provide exercise program modifications and updates. Furthermore, it will be understood that various other types of tools, features, and information can be provided to the user via the website or mobile application.

From the foregoing, it will be seen that the measurement and presentation of the user's health measurements and information can provide the user with the tools to manage his/her weight and overall health. This system can avoid the downsides of temporary health fads by offering actual, measurable information that can be used to form enduring, effective, and improved lifestyle changes. This information can empower the user to managing his/her weight, eat properly, and maintaining an active lifestyle in order to reduce his/her propensity to illness and disease and prolong his/her life expectancy.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A device for monitoring health, the device comprising:
a body scale housing;
one or more sensors disposed within the body scale housing;
a controller coupled to the one or more sensors and configured to determined a user's weight and body mass index, and one or more of a user's blood pressure, body temperature, blood glucose levels, c-reactive protein, ketone levels, oxygen saturation, blood cholesterol levels, testosterone levels, and progesterone levels; and
a communications device coupled to the controller and configured to communicate data from the controller to an external database over a wireless network.

2. The device of claim 1, further comprising a storage device disposed within the body scale housing and coupled to the controller.

3. The device of claim 1, further comprising a near field communication chip.

4. The device of claim 1, wherein the controller is configured to identify one or more of arrhythmia, bradycardia, and atrial fibrillation when experienced by a user of the device.

5. The device of claim 1, wherein the communications device utilizes 3G, GSM, or CDMA wireless communications standard.

6. A system for monitoring health, the device comprising:
a body scale including one or more sensors;
a body attachment including one or more sensors;
a communications link between the body attachment and the body scale;
a controller coupled to the body scale and configured to use one or more measurements from the one or more sensors of the body scale or the body attachment to determined a user's weight and body mass index, and one or more of a user's blood pressure, body temperature, blood glucose levels, c-reactive protein, ketone levels, oxygen saturation, blood cholesterol levels, testosterone levels, and progesterone levels; and
a communications device coupled to the controller and configured to communicate data from the controller to an external database over a wireless network.

7. The system of claim 6, wherein the body attachment is a ring.

8. The system of claim 6, wherein the body attachment is a watch.

9. The system of claim 6, wherein the body attachment is a band.

10. The system of claim 6, wherein the body attachment is an anklet.

11. The system of claim 6, wherein the body attachment is an implanted disposed below the epidermis of a user.

12. The system of claim 6, wherein the body attachment includes a near field communication chip for communicating with another computing device.
